(19) United States
(12) Patent Application Publication
(10) Pub. No.: US 2010/0076378 A1
(43) Pub. Date: Mar. 25, 2010

(54) Safety Medical Syringe with Retractable Needle and Including a Plunger that is Received within a Barrel

(75) Inventor: Vincent Runfola, Apopka, FL (US)

Correspondence Address: EDELL, SHAPIRO & FINNAN, LLC, 1901 RESEARCH BOULEVARD, SUITE 400, ROCKVILLE, MD, 20850, US

(73) Assignee: MIDLAND MEDICAL DEVICES HOLDINGS, LLC, Midland, TX (US)

(21) Appl. No.: 12/563,362

(22) Filed: Sep. 21, 2009

Related U.S. Application Data
(63) Continuation of application No. PCT/US2008/057565, filed on Mar. 20, 2008.
(60) Provisional application No. 60/896,068, filed on Mar. 21, 2007.
(60) Provisional application No. 60/910,227, filed on Apr. 5, 2007.

(51) Int. Cl. A61M 5/50

(52) U.S. Cl. 604/110

(57) ABSTRACT

A syringe includes a hollow barrel including a proximal end with an opening and a distal end configured to receive a syringe needle, where a portion between the proximal and distal ends of the barrel defines a fluid cavity to receive and expel fluid from the barrel distal end, a plunger extending into the proximal end opening of the barrel and axially movable within the barrel toward and away from the barrel distal end, where the plunger includes a proximal end and a distal end that extends within the barrel and is movable toward and away from the barrel distal end to facilitate drawing fluid into and forcing fluid from the fluid cavity within the barrel. The plunger further includes a thumb pad with a flange that extends transversely from a longitudinal orientation of the plunger and includes a dimension that is greater than the proximal end opening of the barrel, and at least a portion of the flange is resiliently flexible and bendable to permit insertion of the flange within the plunger so as to lock the plunger within the barrel after a single use of the syringe.

### CROSS REFERENCE TO RELATED APPLICATIONS

This application is a continuation of PCT/US2008/057565, filed Mar. 20, 2008, which claims priority from U.S. Provisional Patent Application Ser. No. 60/896,068, filed Mar. 21, 2007, and U.S. Provisional Patent Application Ser. No. 60/910,227, filed Apr. 5, 2007. The disclosures of these patent applications are incorporated herein by reference in their entireties.

### FIELD

The present invention pertains to safety medical syringes and, in particular, to syringes that are limited to a single use.

### BACKGROUND

Retractable and/or other types of single use syringes have become extremely increasingly important and desirable for use in hospitals and medical facilities for a number of reasons. In retractable syringes, a hypodermic needle of the syringe is typically withdrawn into the syringe after aspiration and injection of a fluid, thus preventing multiple uses of the syringe and the potential transmission of human immunodeficiency virus (HIV) as well as other diseases from patient-to-patient. The retraction of the needle within the syringe after use also shields the needle and prevents inadvertent needle jabs or pricks from occurring to patients and health care providers.

A variety of different retractable or other types of single use syringe devices have been designed to effectively withdraw the needle within the syringe after use. However, the challenge exists to design a single use syringe that is simplistic in design and assembly, such that the syringe can be produced on a large production scale while minimizing manufacturing costs.

### SUMMARY OF THE INVENTION

A syringe comprises a hollow barrel including a proximal end with an opening and a distal end configured to receive a syringe needle, where a portion between the proximal and distal ends of the barrel defines a fluid cavity to receive and expel fluid from the barrel distal end, a plunger extending into the proximal end opening of the barrel and axially movable within the barrel toward and away from the barrel distal end, where the plunger includes a proximal end and a distal end that extends within the barrel and is movable toward and away from the barrel distal end to facilitate drawing fluid into and forcing fluid from the fluid cavity within the barrel. The plunger further includes a thumb pad with a flange that extends transversely from a longitudinal orientation of the plunger and includes a dimension that is greater than the proximal end opening of the barrel, and at least a portion of the flange is resiliently flexible and bendable to permit insertion of the flange within the plunger so as to lock the plunger within the barrel after a single use of the syringe.

The above and still further objects, features and advantages of the present invention will become apparent upon consideration of the following detailed description of a specific embodiment thereof, particularly when taken in conjunction with the accompanying drawings wherein like reference numerals in the various figures are utilized to designate like components.

### BRIEF DESCRIPTION OF THE DRAWINGS

FIG. 1 is a side view in cross-section of a syringe in accordance with a further embodiment of the present invention, where the needle extends from the syringe barrel and the syringe is ready for use.

FIG. 2 is a partial view in perspective and partial section of the syringe of FIG. 1 including a proximal end portion of the syringe barrel.

FIG. 3 is an exploded view in perspective of a portion of the syringe of FIG. 1 including the needle assembly and a distal end portion of the syringe barrel with a section removed to reveal the barrel interior.

FIGS. 4-7 are partial side views in cross-section of the syringe of FIG. 1 detailing interaction of the distal end of the plunger and the proximal end of the needle assembly at varying stages of depression of the plunger to facilitate retraction of the needle assembly into the syringe in accordance with the present invention.

FIG. 8 is a side view in cross-section of the syringe of FIG. 1 with the needle fully retracted into the syringe after use.

FIG. 10 is a partially exploded side view in cross-section of a portion of another embodiment of a syringe in accordance with the present invention.

FIGS. 10A and 10B are alternative embodiments of a thumb pad for the plunger of the syringe of FIG. 10.

FIGS. 10C and 10D are partial side views in cross-section of a syringe including another embodiment of a thumb pad for the syringe.

FIG. 10E is a plan view of the thumb pad proximal end including a top or proximal end surface of the thumb pad for the embodiment of FIGS. 10C and 10D.

FIGS. 10F and 10G are plan views of different plunger embodiments in which the thumb pad of the plunger includes different shaped and/or patterned grooves on a top or proximal end surface of the thumb pad.

### DETAILED DESCRIPTION

In accordance with the present invention, a single use syringe is provided in which a plunger is completely inserted into a barrel of the syringe and prevented from being removed due to a thumb pad portion of the plunger flexing to frictionally engage with the barrel as the thumb pad is inserted within the barrel. An exemplary embodiment of a single use retractable syringe incorporating the plunger thumb pad is described below. However, it is noted that the invention is not limited to this exemplary embodiment, but could be incorporated into any retractable syringe and/or other single use syringe device.

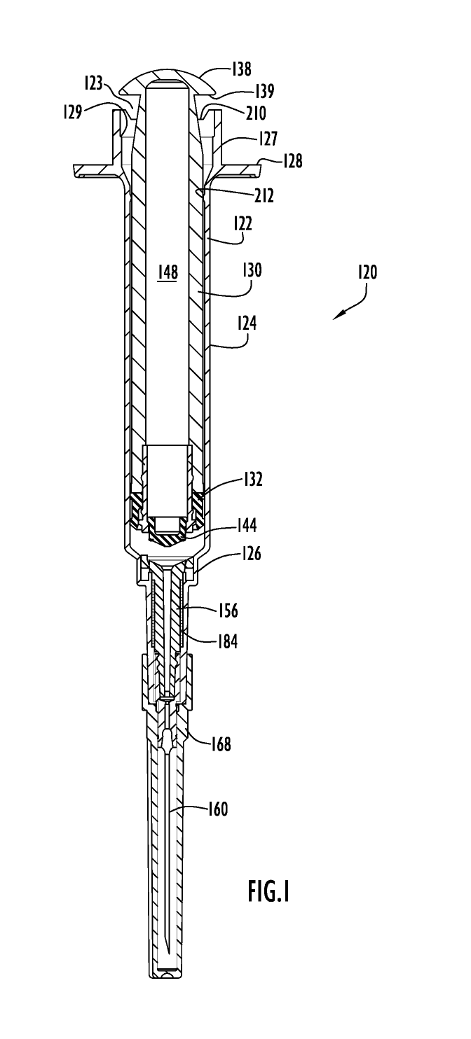

Referring to FIG. 1, a medical syringe 120 includes a hollow cylindrical barrel 122 with an opening 123 at its proximal end and suitably dimensioned to receive a hollow plunger 130. The barrel further includes an opening at its distal end to permit exposure of a needle 160 from the syringe. The barrel 122 includes a main body portion 124 that receives and retains a portion of the plunger 130 and a distal end extension 126 of reduced internal diameter in relation to the main body portion 124 that receives a needle assembly 156 as described below. At least one bump or ridge 212 is disposed along an interior surface wall portion of the main body portion 124 within and near the proximal end of the barrel to resist or prevent complete removal of the plunger from the barrel during operation of the syringe. For example, the ridge could be continuous (e.g., forming a protruding annular ring) within the barrel. Alternatively, a series (e.g., two or more) of ridges can be provided at radially spaced locations along the interior barrel surface.

The plunger 130 includes a resilient seal 132 encircling the plunger near its distal end. A fluid cavity 150 is defined within the barrel 122 between the resilient seal 132 and other distal end portions of the plunger and a distal end 125 of the barrel main body portion 124, where the fluid cavity varies in volume based upon axial displacements of the plunger with respect to the barrel. A sheath 168 is removably secured to the distal end of the barrel 122 to enclose needle 160 secured within the barrel prior to use of the syringe. As in the previous embodiments, the barrel, plunger, resilient seal, sheath and all other components of the syringe may be constructed of any suitable medical grade materials (e.g., plastics and/or stainless steels) that facilitate operability of the syringe as described below. Further, the syringe can be designed with a suitable fluid cavity to meet any fluid volume capacity for a particular application (e.g., 1 cubic centimeter or cc, 3 cc, 5 cc, etc.).

The proximal end of plunger 130 includes a convex surface or domed thumb pad 138 and a radially extending flange 139 that facilitates engagement with the fingers and/or thumb of the user during operation of the syringe. Similarly, the main body portion 124 of the barrel includes a radially extending flange 128 disposed near its proximal end for facilitating engagement with the fingers and/or thumb of the user during operation. An extended barrel portion 127 extends between flange 128 and the proximal end of the barrel and is slightly greater in internal diameter in comparison to the remainder of main body portion 124. The extended barrel portion 127 is also of a sufficient longitudinal dimension, and is slightly smaller in internal diameter than the transverse dimension of the plunger defined at the flange 139. When the plunger 130 is fully depressed within the barrel, an outer peripheral portion of the plunger flange 139 flexes or bends slightly in a manner as described below to permit the flange to enter the extended barrel portion.

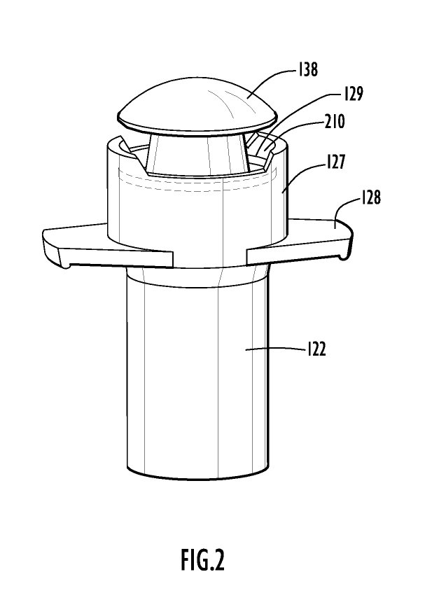

Optionally, two diametrically opposed sections can be removed from extended barrel portion 127, as shown in FIG. 2, so as to form cut-out areas or portions 210 along the extended barrel periphery. The cut-out portions 210 facilitate exposure of sufficient portions of the plunger thumb pad 138 and flange 139 to assist the user of the syringe in gripping the plunger when the plunger is pulled from the barrel to initiate aspiration of fluid within the fluid cavity of the barrel as described below. While two cut-out sections are depicted, it is noted that any suitable number of cut-out sections can be provided (e.g., one or more) at any one or more selected locations along the extended barrel portion. In addition, the extended barrel portion wall thickness and/or plastic or other materials from which this portion is formed can be selected to facilitate a slight elastic and reversible deformation of the extended barrel portion when grasped by a user so as to further assist in axially displacing a portion of the plunger from the barrel during aspiration of fluid into the barrel. However, it is noted that no cut-out sections are required to facilitate forcing of the plunger thumb pad within the barrel in the manner described below.

An annular groove 129 is disposed along interior wall sections (e.g., wall sections that are separated by the cut-out sections) of the extended barrel portion near the proximal end of the barrel. Upon complete depression of the plunger within the barrel and retraction of the syringe within the plunger, the plunger flange 139 engages in a snap-tight locking relationship with annular groove 129 to prevent removal of the plunger from the barrel.

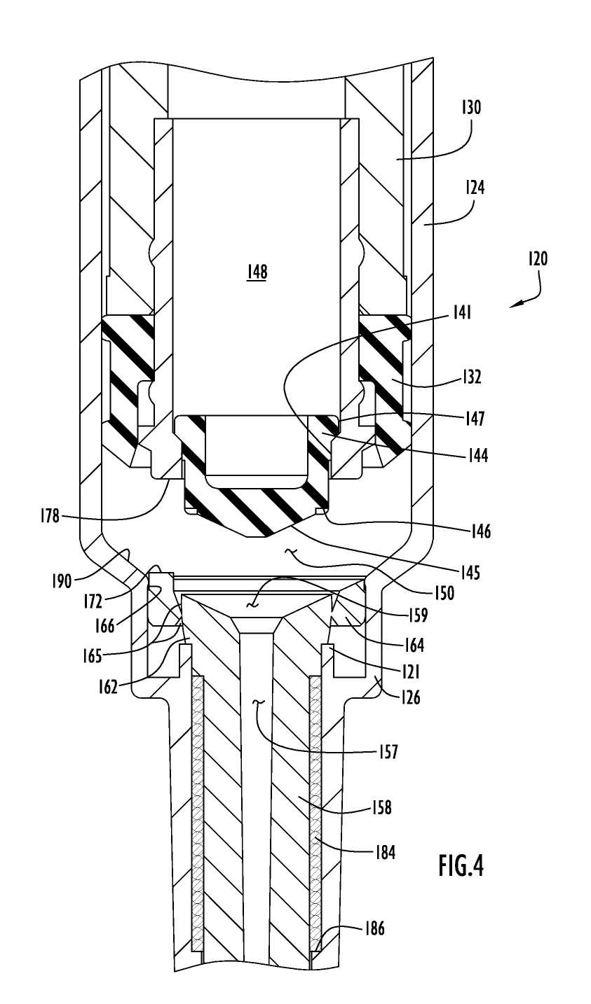

Referring to FIG. 4, plunger 130 includes a membrane or plug 144 disposed at a distal end of the plunger and that is frictionally held between interior wall portions of the plunger. The plug 144 seals the hollow interior or retraction cavity 148 of plunger 130, with the frictional engagement between the plug and the plunger being suitable to maintain engagement of the plug with the plunger until retraction of the needle assembly occurs as described below. Alternatively, it is noted that the plunger membrane or plug can be secured at the distal end of the plunger in a snap tight fitting relationship or in any other suitable manner.

As can be seen in FIG. 4, the distal end of plunger 130 includes an opening that communicates with retraction cavity 148 and into which plug 144 is secured. The interior annular wall at the distal end of the plunger includes an inwardly extending radial ledge 141. The diameter or transverse cross-section of the plunger opening, as defined at the ledge 141, is smaller than the diameter or transverse cross-section of the retraction cavity 148 that is defined within the plunger and lies beyond the ledge 141. Similarly, plug 144 includes an extending portion 147 that extends transversely from a proximal end of the plug. The extending portion 147 of the plug 144 is slightly larger in transverse cross-sectional dimension than the transverse cross-sectional dimension of the plunger opening defined at the ledge 141, so as to facilitate frictional contact between the ledge 141 and the extending portion 147 of the plug 144. The transverse cross-sectional dimensions of the extending portion 147 as well as the rest of the plug 144 are smaller than the transverse cross-sectional dimension of the retraction cavity 148 between the plunger ledge 141 and the proximal end of the plunger. Further, the transverse cross-sectional dimension of the remaining plug portion that extends between the extending portion 147 and the distal end of the plug 144 is slightly smaller than the transverse cross-sectional dimension of the plunger opening. Thus, upon axial displacement of the extending portion 147 of the plug 144 from the ledge 141 of the plunger 130 during retraction of the needle assembly, the frictional engagement between the plunger and the plug is released and the plug is free to move into the retraction cavity.

The plunger plug 144 includes a convex and frusto-conical surface 145 that extends toward the distal end of the barrel and engages with a generally complimentary, concave and frusto-conical cavity portion 159 of needle assembly 156 when the plunger is fully depressed into the barrel as described below. As noted in the previous embodiments described above, the end wall may alternatively be formed with any suitable outwardly or inwardly extending surface (e.g., conical, convex, V-shaped, multifaceted, etc.) or even a flat or planar surface as desired for a particular application. However, complimentary engaging surface features of the plunger plug and the needle assembly are preferred in order to minimize or eliminate open or “dead” space within the fluid cavity during removal of fluid from the fluid cavity.

At least one notch 146 is formed on the plug at the base of the frusto-conical surface. The notch 146 can extend around the periphery of the plug at the base of the frusto-conical surface or, alternatively, consist of a single notch or one or more spaced notch sections. The notch basically serves to provide a fluid flow path between the fluid cavity within the barrel and the fluid channel in the needle assembly when the plunger is substantially or completely engages with the needle assembly. In addition, the notched plunger plug minimizes or eliminates the potential for an increase or build-up of hydraulic pressure within the fluid cavity during movement of the plunger toward the needle assembly.

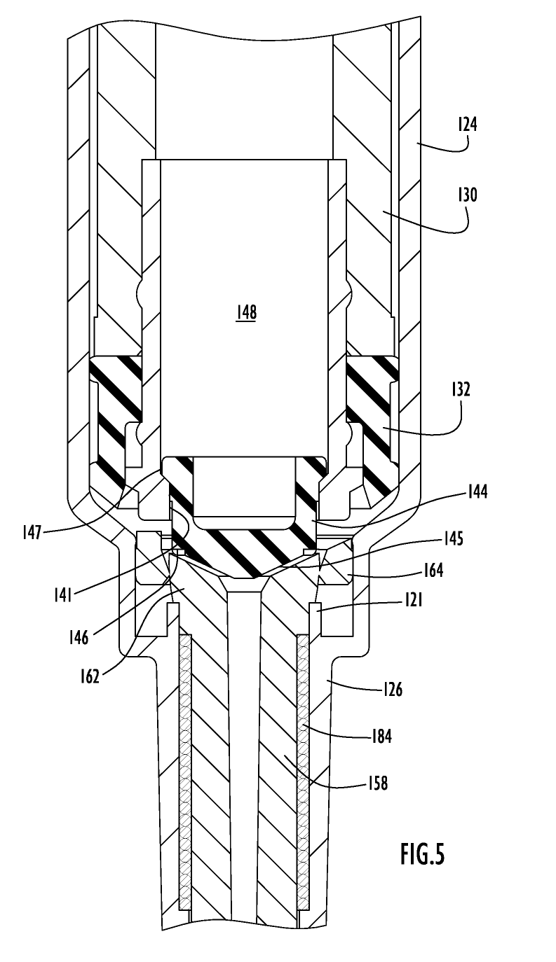

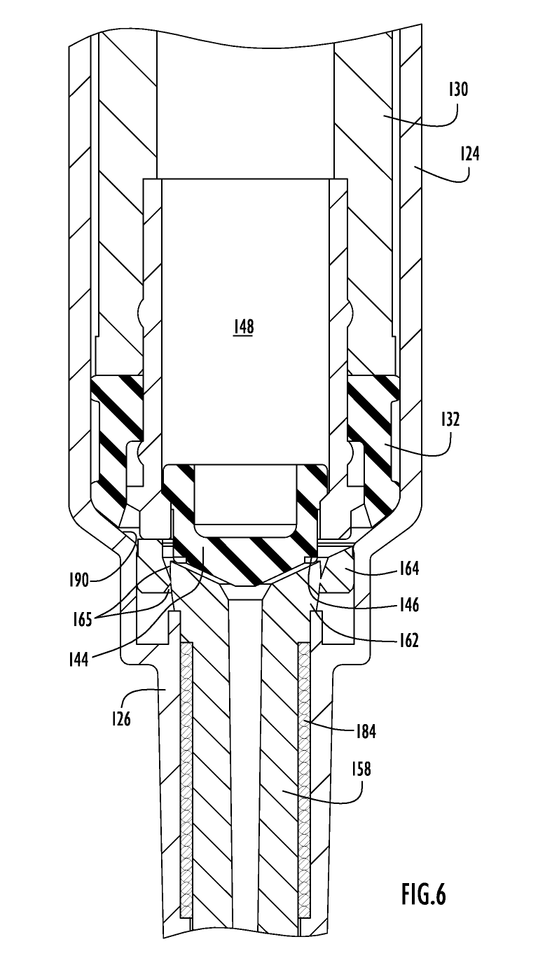

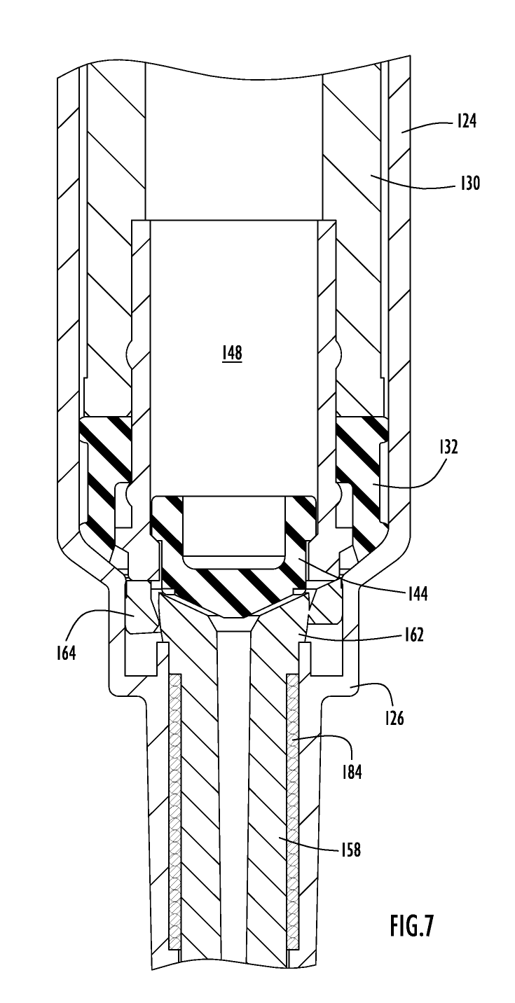

Needle assembly 156 includes a needle holder or stem 158 that connects with a syringe needle 160 and is affixed within the distal end extension 126 of the barrel such that the needle 160 extends from the distal end of the barrel prior to and during use. The needle stem 158 and needle 160 preferably releasably engage with each other via any suitable fluid tight engagement. In a preferred embodiment, the releasable engagement between the needle stem and the needle is a threaded engagement, where the needle stem includes a male threaded configuration and the needle includes a female threaded connector to releasably connect with the needle stem. This connection, which is depicted in FIGS. 1 and 4-7, differs from conventional syringe needle connections, such as Luer Lock configurations. In addition, this threaded configuration provides an easy, universal connection with needles of various gauges and types. However, it is noted that the needle can also be attached to the needle stem in any other suitable releasable or non-releasable manner.

A cavity 157 extends axially from a proximal end of the needle stem 158 to the connection point with the needle 160 in order to facilitate fluid communication between the needle and fluid cavity 150 within the barrel. In addition, cavity 157 includes a widened portion 159 at the proximal end of needle stem 158 that is frusto-conical in configuration and widens toward the proximal end of the barrel so as to be generally aligned and complimentary with the frusto-conical surface 145 of the plunger 130. As noted above, when the plunger is depressed toward the needle assembly, the frusto-conical surface 145 of plug 144 generally aligns and engages with widened portion 159. When the plunger plug is brought toward and fully engages widened portion 159 of the needle assembly, notch 146 defined on plug 144 ensures a fluid flow path exists between fluid cavity 150 and needle assembly cavity 157 and reduces or eliminates any build-up of hydraulic pressure within the fluid cavity.

The needle stem 158 further includes a radially extending flange 162 at its proximal end that is suitably dimensioned to engage with a step or ledge 121 disposed along an interior surface of the distal end extension 126 in order to prevent movement of the needle assembly distally beyond ledge 121 during depression of the plunger toward the distal end of the barrel.

A stem ring 164 is secured to and extends radially from the flange 162 of needle stem 158 to engage with the interior wall surface of distal end extension 126 of the barrel. The stem ring 164 is preferably formed or molded as a part of flange 162 and needle stem 158 and includes notched or scored sections 165 at the connection point of stem ring 164 and needle stem 158, where the scored sections are formed on opposing surfaces of both the needle stem flange and the stem ring. The scored sections 165 define a thin membrane or reduced material section that is torn or broken during operation of the syringe to facilitate retraction of needle assembly 156 in the manner described below. The stem ring 164 is preferably dimensioned to facilitate a partial sliding of a broken portion of the stem ring along the interior wall surface of the barrel when the plunger is depressed to engage with needle stem 158 as described below.

The diameter of the stem ring can be selected to be slightly smaller, the same size, or slightly larger than the diameter of the interior wall surface of distal end extension 126 at the location where the stem ring engages the barrel. In the present embodiment, the diameter of stem ring 164 is slightly larger in comparison to the diameter of the interior wall surface of the barrel that engages with the stem ring such that the stem ring is slightly compressed during engagement with the barrel and forms an effective fluid tight seal. The dimensions of the stem ring are further selected to provide a compression fit/fluid tight seal at the stem ring/barrel interior wall interface while facilitating a sliding of the stem ring with the barrel interior wall surface when the plunger is completely depressed within the barrel. Alternatively, it is noted that the stem ring can be connected directly to the barrel interior wall surface (e.g., via adhesive bonding, welding, etc.).

A radial protrusion or annular shoulder 166 is disposed along the interior surface and near the proximal end of the distal end extension 126 of the barrel. The shoulder 166 engages with stem ring 164 to prevent movement of the needle assembly toward the proximal end of the barrel while the stem ring remains attached with needle stem 158. A resilient member 184 (e.g., a coil spring) is disposed between the flange 162 of needle stem 158 and an interior ledge 186 disposed on the interior surface of the distal end extension 126 at a location between ledge 121 and the distal end of the barrel. When the needle assembly 156 is press fit into the distal end extension 126 of the barrel (as described below) such that stem ring 164 is extended distally beyond the radial protrusion 166 of the barrel, resilient member 184 is compressed to bias the needle assembly toward the proximal end of the barrel.

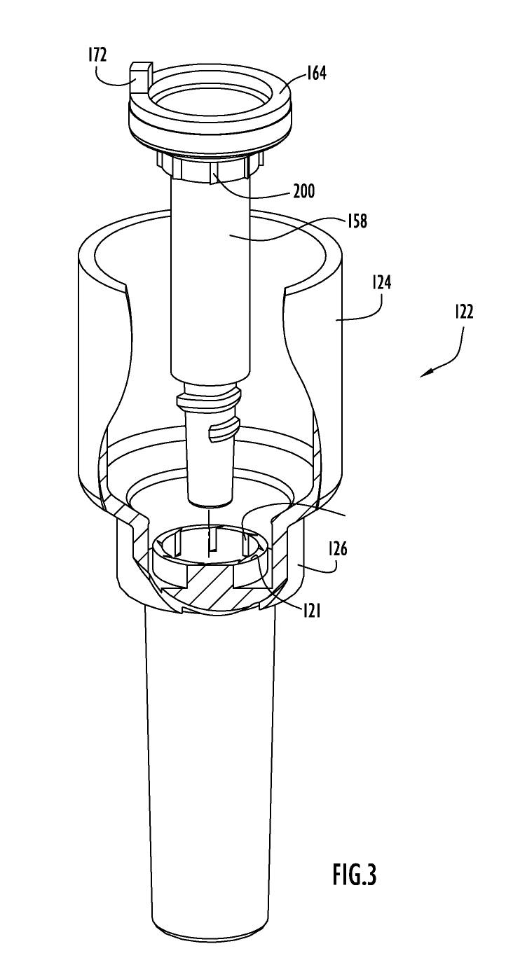

Referring to FIG. 3, the needle stem and barrel include complimentary protrusions and grooves that facilitate easy assembly of the needle stem within the barrel and further limit or prevent any rotation of the needle stem with respect to the barrel after assembly and during operation of the syringe. In particular, needle stem 158 includes a set of protrusions or teeth 200 extending transversely from the outer surface of the needle stem at a location proximate the stem ring 164. The teeth are spaced around the periphery of the needle stem and are aligned to correspond with complimentary grooves 202 disposed along the interior barrel wall surface at the distal end extension 126 and at a location generally corresponding with the barrel ledge 121. As can be seen from FIG. 3, the teeth 200 and complimentary grooves 202 are configured to facilitate easy assembly of needle stem 150 within barrel 122, where the needle stem rotates as necessary upon insertion within the barrel until the teeth are appropriately aligned with the grooves and the needle stem is forced in place within the barrel distal end section. Once needle stem 158 is assembled within barrel 122 and the teeth 200 engage with the grooves 202, the needle stem is prevented from rotating with respect to the barrel during operation of the syringe (e.g., when attaching or removing a needle from the needle stem, and during retraction of the needle assembly within the plunger).

Alternatively, the syringe can be provided with other needle stem and barrel configurations that provide a locking engagement between the needle stem and the barrel. For example, the needle stem and barrel can include suitable complimentary protrusions and/or grooves to achieve a ratcheting or cam configuration that permits a selected degree of rotational movement of the needle stem within the barrel in one direction while limiting or preventing rotational movement of the needle stem within the barrel in an opposite direction.

The syringe 120 is designed so that complete depression of plunger 130 within barrel 122 facilitates a displacement of plunger plug 144 from the plunger and also a tearing or breaking away of the stem ring 164 from flange 162 of needle stem 158 to facilitate retraction of the needle stem and the needle 160 into the retraction cavity 148. In this embodiment, stem ring 164 of needle stem 158 includes at least one raised ridge 172 that extends from a proximal end of the needle stem and is configured to make initial contact with annular distal edge 178 of the plunger when the plunger is displaced within the barrel. While only one ridge 172 is depicted in FIGS. 4-7, it is noted that any selected number of ridges can be provided at any suitable locations and suitably spaced from each other along the proximal end of the needle stem that faces the plunger. The annular distal edge 178 of the plunger is generally planar in configuration. However, it is noted that the plunger distal end can include any one or more protrusions like the needle stem or, alternatively, have an angled contour to form an apex.

Plunger resilient seal 132 is further designed and suitably dimensioned and positioned around the plunger proximate the plunger distal end such that, upon complete depression of the plunger within the barrel, a distal end of the resilient seal engages and compresses slightly against a narrowing portion 190 of the interior barrel wall that defines a transition between main body portion 124 and distal end extension 126. The resilient seal does not slide with respect to the plunger, but rather compresses slightly against the barrel wall narrowing portion at the end of the plunger stroke that initiates needle retraction, and this serves to further minimize or eliminate “dead” space within the fluid cavity 150 as well as to displace any residual fluid from the fluid cavity into needle stem cavity 157.

The needle stem can be assembled within the syringe such that the ridge (or ridges) is aligned in any selected orientation with respect to the plunger distal end. The stem ring ridge(s), plunger and annular distal end of the plunger and needle stem are suitably dimensioned in the longitudinal direction of the syringe and further suitably aligned with each other to facilitate engagement of stem ring ridge(s) 172 and/or other proximal end surface portions of stem ring 164 with annular distal edge 178 of the plunger and plunger plug 144 with needle stem 158 upon complete depression of the plunger, which in turn facilitates a tearing or breakage of stem ring 164 from needle stem flange 158 at the scored sections 165 and a forcing of plunger plug 144 from its frictional engagement with plunger ledge 141 to initiate retraction of needle assembly 56 along with the plunger plug into retraction cavity 48 of the plunger.

Assembly of the syringe is achieved in a similar manner as the other syringe embodiments described above. Resilient member 184 and then needle assembly 156 (with or without the needle 160) are first inserted into opening 123 at the proximal end of barrel 122, through main body portion 124 and into the distal end extension 126. As the stem ring 164 of needle stem 158 encounters annular shoulder 166 of the barrel, the stem ring is compressed slightly and forced distally beyond the shoulder 166 in a snap-fit engagement. Once stem ring 164 is forced distally beyond shoulder 166, the needle assembly 156 is locked in place within the distal end extension 126, and resilient member 184 is compressed to bias the needle assembly proximally within the syringe. The stem ring 164 remains compressed to a selected degree between flange 162 and the barrel interior wall surface in this locked configuration and provides an effective fluid tight seal at its compressed fit contact point with the barrel.

Plunger 130 is assembled by inserting plunger plug 144 into the plunger such that the extending portion 147 of the plug frictionally engages plunger ledge 141 located at the open distal end of the plunger and a distal portion of the plunger plug (including plug surface 145 and notch 146) extends from the plunger. The plunger plug can be inserted directly into the open distal end of the plunger to its frictional fitting position. In the embodiment depicted in FIGS. 1-8, the proximal end of the plunger, including thumb pad 138 and flange 139, is a single molded or formed part. Alternatively, in embodiments where the plunger flange and thumb cap are separate from the rest of the plunger (as in the embodiment described below and depicted in FIG. 9), the plunger plug can first be inserted through an open proximal end of the plunger and pushed into its frictional fitting position prior to sealing this open end with the flange and thumb pad.

After assembly of the plunger, the plunger is inserted into opening 123 of barrel 122 and is axially displaced a suitable distance to facilitate use of the syringe. The needle 160 may be connected with needle stem 158 prior to insertion of the needle assembly into barrel 122 (e.g., via the threaded engagement as depicted in FIG. 3). Alternatively, needle 160 may be connected with the connecting portion of needle stem 158 after securing the needle stem within the barrel. Once secured, needle 160 protrudes from the opening at the distal end of the barrel after assembly to facilitate use of the syringe.

In operation, the distal end of the plunger is displaced a suitable distance toward the proximal end of the barrel to draw fluid from needle 160 into fluid cavity 150. Upon injection of the needle into an injection site, the plunger is then depressed toward the distal end of the barrel to force fluid from cavity 150 and through needle 160. Referring to FIGS. 4-7, as the plunger is further depressed within the barrel, the frusto-conical surface 145 of plunger plug 144 moves into the widened portion 159 of central cavity 157 of needle stem 158 to force any remaining fluid through the needle prior to retraction (thus reducing “dead” space between the engaging portions of the plunger and needle assembly). The notch 146 on the plunger plug provides a fluid channel for fluid to continue to flow into central cavity 157 even when plunger surface 145 is substantially in contact with the widened cavity portion 159. In addition, ridge(s) 172 of needle stem 158 initially engages a portion of annular distal edge 178 of the plunger to initiate a tearing or breaking away of stem ring 164 from needle stem 158 along the scored sections 165. Approximately contemporaneously, surface 145 of plunger plug 144 engages needle stem 158 within the widened cavity portion 159, and such continued depression of the plunger toward the needle assembly overcomes the frictional force holding the plunger plug within the plunger, forcing the plunger plug toward the proximal end of the plunger and into retraction cavity 148.

Complete depression of the plunger within the barrel further forces plunger annular distal edge 178 against other surface portions of stem ring 164, causing the portion of the stem ring that has already broken away from flange 62 to slide distally a short distance along the interior wall of the barrel so as to become oriented at a slight angle with respect to the central axis of needle stem 158. In addition, the portion of the stem ring that has not broken away is prevented from moving distally until it has become broken away from the needle stem. This sliding of the broken portion of the stem ring 164 along the interior wall of the barrel, in combination with the continued pressure applied by the fully depressed plunger to the stem ring, results in a progressive tearing or breakage of the stem ring in both directions along scored sections 165 until the stem ring is fully separated from tab 162. In addition, the forced engagement of plunger plug 144 with needle stem 158 results in further movement of the plunger plug from its frictional engagement with the distal end ledge 141 of the plunger, resulting in dislodging of the plunger plug from the plunger. Plunger resilient seal 132 compresses slightly against the narrowed portion 190 of the barrel interior wall to force residual fluid into the needle assembly, while plunger plug notch 146 provides a fluid flow path for such fluid when plunger surface 145 engages with needle stem 158 within widened cavity portion 159.

The design of the syringe can be configured such that the stem ring 164 is completely separated from needle stem 158 immediately prior to, substantially simultaneously with, or immediately after the complete dislodging of plunger plug 144 from the plunger. As shown in the series of FIGS. 4-7, the initial dislodging and axial displacement of plunger plug 144 begins just prior to initial contact between stem ring ridge 172 and plunger distal edge 178 and partial breakage of stem ring 164 from needle stem tab 162. However, the syringe can also be designed such that initial and partial breakage of the stem ring occurs prior to any initial dislodging and axial displacement of the plunger plug. Once complete separation of the stem ring from the tab of the needle stem and at least a partial dislodging of the frictional engagement between the plunger plug and the plunger is achieved, resilient member 184 forces needle stem 158 and needle 160, along with plunger plug 144, proximally into the retraction cavity 148 within the plunger.

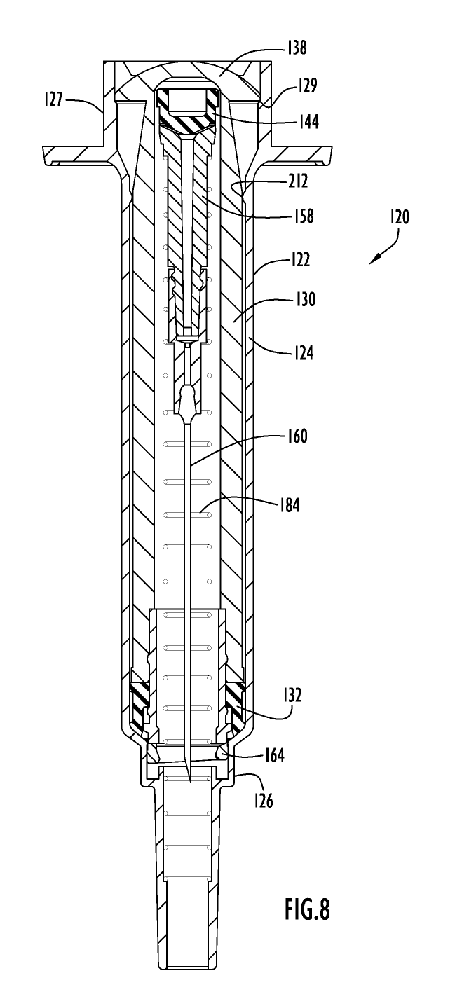

As can be seen from FIG. 8, when plunger 130 has been fully depressed within barrel 122 and retraction of needle assembly 156 has occurred, flange 139 of the plunger extends slightly into the extended barrel portion 127 and is locked within annular groove 129. As noted above, an outer peripheral portion (or outer peripheral portions) of the plunger thumb pad flange flex or bend slightly upward (toward the barrel proximal end) as the flange is inserted within the barrel and locked within the annular groove. In this locked position, removal of the plunger from the barrel is prevented.

The plunger can include a thumb pad or cap that is a separate part or component from the main plunger body. An exemplary embodiment is depicted in FIG. 9. Syringe 220 includes a barrel 122 and needle assembly that are substantially similar to the same parts or components as described above and depicted in FIGS. 3-8. The plunger includes a hollow main body portion 230 including a resilient seal 232 disposed near the distal end of the main body portion and that is substantially similar to the plunger seal described above in the previous embodiment. A thumb pad or cap 238 includes an insert 242 that is suitably dimensioned to fit within and frictionally engage with an opening defined at the proximal end of the main body 230. The thumb cap further includes a flange 239 that is of greater transverse cross-sectional dimension than the insert 242 and abuts the proximal end of the plunger upon engagement of the insert within the plunger. Thumb pad 238 further provides a gripping surface for the plunger during use of the syringe.

An annular edge portion 240 is formed at the periphery of flange 239 (i.e., an outer peripheral portion of the flange). The annular edge portion 240 is constructed of a suitable material, is suitably dimensioned and/or includes any other suitable structure to permit resilient flexing and bending of the annular edge portion with respect to the remaining portion of the flange 239 (e.g., bending of the annular edge portion by as much as 45°, 60°, 90° or greater with respect to the remaining portion of the flange). For example, the annular edge portion can be formed of a material having a different hardness or durometer value (e.g., measured as a Shore A or Shore D durometer value) in relation to the remaining portion of the flange, where the annular edge portion is a softer material than the remaining portion of the flange, to permit such flexure of the edge portion with respect to the flange.

Alternatively, or in addition to the annular edge portion being formed of a different material in relation to the flange, the annular edge portion can have a different thickness, different dimensions and/or other structural differences in relation to the remaining portion of the flange to permit such flexure and bending of the edge portion. As shown in FIG. 9, the flange 239 tapers to the annular edge portion 240, such that the edge portion 240 has a reduced thickness in relation to other portions of the flange that are closer in proximity to insert 242. This reduced thickness can be selected so as to permit suitable flexure of the annular edge portion with respect to the remaining portion of the flange. In addition, one or more scored sections can be provided along the flange at suitable radial distances from insert 242, where the one or more scored sections provide a flexure or bending point or location (e.g., an “artificial hinge” or “living hinge”) for the annular edge portion 240.

The resilient flexing and bending features of the annular edge portion 240 permits edge portion to bend upward or toward a proximal end portion 251 of the thumb pad while sliding against an internal peripheral wall portion 252 of barrel portion 127 when the plunger with thumb pad are received within the extended barrel portion (i.e., during or upon completion of retraction of the needle within the plunger retraction cavity). Upon reaching annular groove 129 within barrel portion 127, edge portion 240 flexes back to its original, unflexed position so as to fit within and engage groove 129, thus locking the plunger within the barrel to prevent or significantly frustrate efforts to remove the plunger from the barrel. At this locked position (i.e., with edge portion 240 being disposed within barrel annular groove 129), the thumb pad 238 is nearly or substantially entirely received within extended barrel portion 127 (e.g., with the proximal end of the thumb pad being generally flush or co-planar or slightly recessed with respect to the proximal end of the barrel).

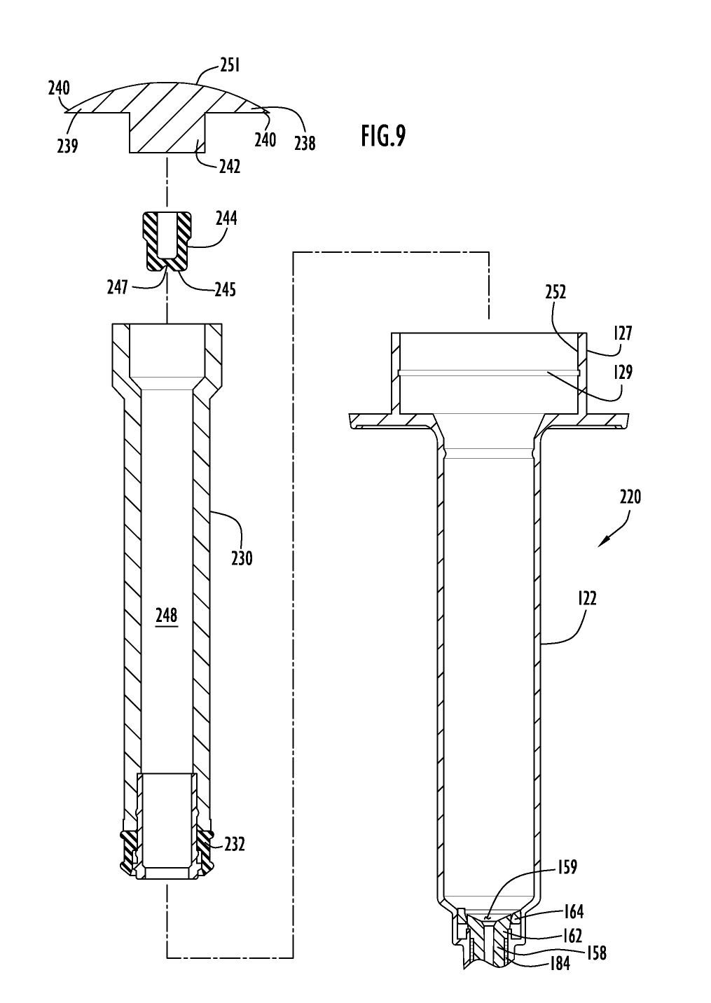

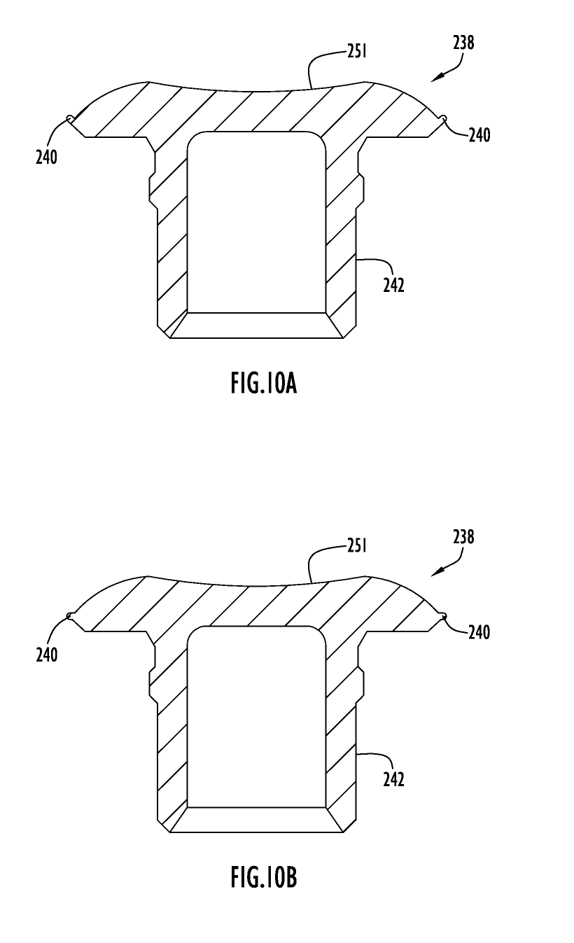

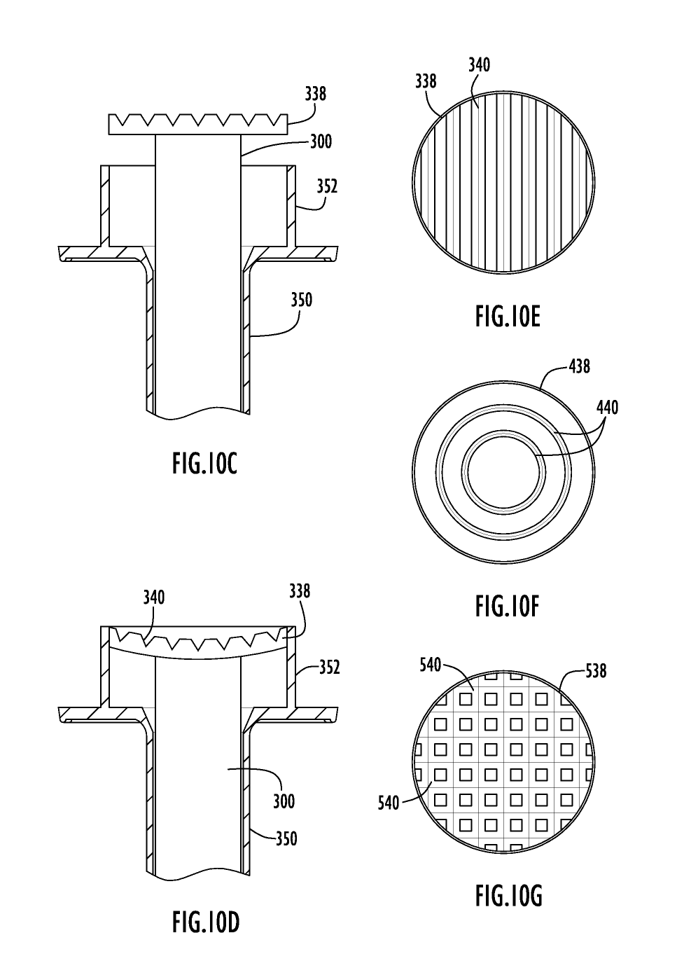

FIGS. 10A and 10B show alternative embodiments of a thumb pad for use with the plunger. In FIG. 10A, the annular edge portion 240 includes a slightly angled surface (e.g., an annular edge portion that is angled upward toward the proximal end portion of the thumb pad at an angle from about 30° to about 60° or greater in relation to a plane that is perpendicular to a longitudinal axis of the thumb pad), while the edge portion 240 in FIG. 10B includes relatively flat surfaces surrounding the edge portion (i.e., the edge portion includes surfaces that are substantially parallel in relation to a plane that is perpendicular to a longitudinal axis of the thumb pad). In each embodiment, the edge portion is configured to flex or bend slightly upward (toward the proximal end portion of the thumb pad) so as to slide against the internal barrel wall when the plunger and thumb pad are forced distally into the opening at the proximal end of the barrel until the edge portion engages with the annular groove (thus flexing back to its original, relaxed position).

A plunger plug 244 is frictionally held within an opening at the distal end of the plunger in a substantially similar manner as described in the previous embodiment. However, plug 244 differs from the plunger plug of the previous embodiment in that it includes a generally flat or planar distal end surface 245 with a notch 247 disposed along this surface. The planar surface 245 of plug 244 engages with a portion of the widened cavity portion 159 of the needle stem 158, while notch 247 serves to minimize hydraulic pressure build-up within the syringe cavity during axial movement of the plunger toward the needle assembly to initiate needle retraction.

The design of the plunger in this manner allows easy assembly of the plunger plug. In particular, plug 244 can be inserted within plunger main body 230 at the open proximal end prior to connection of thumb cap 238 with the main body. The plunger plug can be forced through the plunger retraction cavity 248 into frictional engagement at the open distal end of the plunger main body, followed by insertion of thumb cap insert 242 into the opening at the plunger proximal end. Thumb cap 238 can be firmly secured to main body 230 in any suitable manner (e.g., via an adhesive, welding, etc.) to prevent removal of the thumb cap from the main body after assembly. Once the plunger is assembled within barrel 122, the syringe 220 operates in a substantially similar manner as the previous embodiment described above and depicted in FIGS. 3-8 to facilitate a single use and needle retraction, with the additional feature of the thumb pad annular edge portion 240 flexing to permit entry of the thumb pad flange 239 within the barrel and to lock edge portion 240 within the annular groove 129 in the barrel after sufficient displacement of the plunger within the barrel.

FIGS. 10C-10G depict further embodiments of a syringe including a plunger with a thumb pad disposed at its proximal end that is of greater transverse cross-sectional dimension (e.g., diameter) than the opening at the extended barrel portion. In these embodiments, the plunger thumb pad and plunger form a single molded piece. However, it is noted that any of these and the other flexible plunger thumb pad flange embodiments can be formed on a plunger thumb pad or cap that is a separate component or piece from the main plunger body or, alternatively, formed integrally (e.g., as a single molded piece) with the main plunger body.

In the embodiment of FIGS. 10C and 10D, thumb pad 338 of plunger 300 includes a series grooves 340 formed within the proximal or top surface of the thumb pad and arranged in rows with respect to each other so as to extend between diametrically opposed peripheral sections of the thumb pad. The grooves taper to form a general “V” shape in a cross-section of the grooves (i.e., in a depth dimension of the grooves). However, it is noted that the grooves can have any selected cross-sectional shape and/or be aligned in any selected manner across the top surface of the thumb pad. The grooves are formed in the thumb pad to permit bending or flexing of the thumb pad during insertion of the thumb pad into the extended barrel portion opening.

As can be seen in FIGS. 10C-10E, the thumb pad 338 bends or flexes as the thumb pad is received within the extended barrel portion 352 of barrel 350, so as to permit the peripheral edge portions of the thumb pad to frictionally engage and slide against the internal peripheral wall portions of the barrel during continued movement of the plunger distally within the barrel. The thumb pad 338 bends so as to form a slightly concave contour at its top or proximal end surface, with the center of the thumb pad collapsing to permit the thumb pad to fit within the barrel proximal end opening. The barrel and plunger can be of the same or similar construction and including the same or similar retraction features as the syringes depicted in the previous embodiments. In this embodiment, the extended barrel portion can also include a locking groove to receive and engage with the plunger thumb pad, so as to lock the plunger within the syringe barrel. Alternatively (and as shown in FIGS. 10C and 10D), the barrel can be provided without any annular groove. In such an embodiment (in which there is no annular groove within the barrel), the plunger is still effectively maintained within the barrel once the thumb pad has been received within the extended barrel portion, since the frictional engagement between the thumb pad and the internal peripheral wall portions of the barrel frustrate efforts by a user to remove the plunger.

Any other forms and types of grooves and/or cut-out sections can be provided along the top surface of the plunger thumb pad to permit flexing or bending of the thumb pad when the thumb pad is pressed within the proximal open end of the barrel. FIGS. 10F and 10G show different exemplary embodiments of thumb pad grooves that would permit collapsing or bending of the top surface of the thumb pad in a manner similar to that described above and shown in FIGS. 10C-10E. A series of concentrically aligned and annular grooves 440 are shown on the top surface of thumb pad 438 in FIG. 10F, while FIG. 10G shows a grid of grooves 540 or cut-out sections along the top surface of thumb pad 538 (i.e., grooves extending transversely with respect to each other along the thumb pad top surface). Each of the grooves in the thumb pads of FIGS. 10F and 10G also have a transverse cross section that is “V” shaped. However, as noted above, the grooves can have any suitable cross-sectional geometry.

The present invention is not limited to the syringe embodiments described above and can include additional and/or modified features. For example, as noted above, any of the plunger thumb pad flexible flange portions can be provided on a plunger thumb pad or cap that is a separate piece from the main plunger body (e.g., as shown in FIG. 9) or, alternatively, as a single, integral piece with the main plunger body (e.g., as a single molded piece).

Any suitable plunger thumb pad configuration can be provided which facilitates flexing or bending of one or more outer peripheral or annular flange portions of the thumb pad to facilitate insertion of the plunger thumb pad within a proximal end opening of the syringe barrel while substantially preventing removal of the plunger thumb pad from the barrel (so as to limit the syringe to a single use).

The plunger thumb pad can further be disposed at the proximal end of the plunger or, alternatively, at a suitable location along the plunger that is between the proximal and distal ends of the plunger (preferably closer to the proximal end of the plunger). In embodiments in which the thumb pad is disposed at the proximal end of the plunger (i.e., as shown in FIGS. 1-9), the syringe is preferably designed and configured such that the plunger with thumb pad can be substantially or completely received within the barrel such that an upper or proximal end surface of the plunger thumb pad is substantially flush or coplanar with the barrel proximal end or may even be recessed within the barrel a selected distance from the barrel proximal end.

The syringe is further preferably designed and configured such that, after retraction of portions of the needle assembly with needle into the plunger, the thumb cap can then be forced into (or further into) the barrel to ensure locking engagement with a barrel locking groove or sufficient entry of the thumb pad into the barrel to prevent or frustrate efforts of a user in removing the plunger from the barrel.

The syringe in accordance with the invention can be used with retractable syringe configurations (such as those described above) or, alternatively, in other syringe configurations (e.g., syringes which do not have retractable needle features).

A syringe in accordance with the present invention can also be configured for use in other, needle-free applications (i.e., applications that do not include the use of needles). For example, a syringe of the present invention can be configured with a suitable connector to connect directly with an intravenous (IV) fluid line for injection of fluid from the syringe into the IV line. The retraction of the needle stem would prevent further use of the syringe after a single fluid injection.

While the invention has been described in detail and with reference to a specific embodiment thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

What is claimed:

1. A syringe comprising:
a hollow barrel including a proximal end with an opening and a distal end configured to receive a syringe needle, wherein a portion between the proximal and distal ends of the barrel defines a fluid cavity to receive and expel fluid from the barrel distal end;
a plunger extending into the proximal end opening of the barrel and axially movable within the barrel toward and away from the barrel distal end, wherein the plunger includes a proximal end and a distal end that extends within the barrel and is movable toward and away from the barrel distal end to facilitate drawing fluid into and forcing fluid from the fluid cavity within the barrel, the plunger further including a thumb pad comprising a flange that extends transversely from a longitudinal orientation of the plunger and includes a dimension that is greater than the proximal end opening of the barrel, and at least a portion of the flange is resiliently flexible and bendable to permit insertion of the flange within the plunger so as to lock the plunger within the barrel after a single use of the syringe.

2. The syringe of claim 1, wherein the thumb pad is disposed at the proximal end of the plunger and the plunger and barrel are suitably dimensioned such that, upon complete insertion of the plunger within the barrel, the plunger is completely received within the barrel with a portion of the thumb pad being coplanar with or slightly recessed with respect to the barrel proximal end.

3. The syringe of claim 1, wherein the thumb pad flange includes an annular edge portion that is bendable when the thumb pad is forced within the barrel.

4. The syringe of claim 3, wherein the annular edge portion has a different durometer value in comparison to the durometer value of at least one other portion of the thumb pad flange.

5. The syringe of claim 1, wherein the plunger further comprises a main body portion, and the thumb pad is securable to the main body portion.

6. The syringe of claim 1, wherein the plunger further comprises a main body portion that is molded with the thumb pad as a single, integral piece.

7. The syringe of claim 1, wherein the thumb pad includes at least one groove formed along the flange, wherein the at least one groove defines a reduced material section for the flange that facilitates bending of at least a portion of the flange when the thumb pad is inserted into the barrel proximal end opening.

8. The syringe of claim 7, wherein the at least one groove comprises an annular shaped groove.

9. The syringe of claim 8, wherein the at least one groove comprises a plurality of annular shaped grooves arranged concentrically with respect to each other.

10. The syringe of claim 8, wherein the at least one groove comprises a plurality of linear shaped grooves extending along portions of the flange.

11. The syringe of claim 1, wherein the barrel includes a locking groove that is configured to receive a peripheral end portion of the thumb pad flange of the plunger when the plunger thumb pad is forced within the barrel.

12. The syringe of claim 1, further comprising a retractable needle assembly including a portion that is configured to retract within the syringe after the syringe has been used.

13. A syringe comprising:
a hollow barrel including an opening at a proximal end of the barrel, an opening at a distal end of the barrel and a fluid chamber disposed within the barrel;
a hollow plunger extending into the barrel via the proximal end opening and axially movable within the barrel toward and away from the distal end opening, wherein the plunger includes an end wall that is releasably secured to the plunger at an opening disposed at a distal end of the plunger and is movable from the plunger distal end opening during use of the syringe to facilitate access to a retraction cavity disposed within the plunger; and
a needle assembly secured within and at a distal end of the barrel, the needle assembly including a needle holder, a resilient member biasing the needle holder toward the proximal end opening of the barrel, and a retaining member releasably secured to the needle holder via a reduced material section, wherein the needle holder includes a connector to secure a needle to the needle holder so as to permit the needle to extend through the distal end opening of the barrel;
wherein the plunger, retaining member and needle holder are configured such that the retaining member maintains the needle holder at the distal end of the barrel against the bias of the resilient member and, when the plunger is fully depressed within the barrel, the plunger engages the needle holder to force the end wall of the plunger within the plunger and to force the retaining member to break away and separate from the needle holder along the reduced material section so as to facilitate retraction into the retraction cavity of the end wall, the needle holder and a needle secured to the needle holder; and
wherein the plunger includes a thumb pad disposed at a proximal end of the plunger, the thumb pad including a flange that extends transversely from a longitudinal orientation of the plunger and includes a dimension that is greater than the proximal end opening of the barrel, and an annular edge portion of the flange is resiliently flexible and bendable to permit insertion of the flange within the plunger so as to lock the plunger within the barrel after a single use of the syringe.

14. A method of utilizing a syringe of the type including a hollow barrel including a proximal end with an opening, a distal end and a fluid cavity disposed within the barrel, and a plunger extending into the barrel via the proximal end opening and axially movable within the barrel toward and away from the barrel distal end, the plunger including a proximal end and a distal end that extends within the barrel and is movable toward and away from the barrel distal end to facilitate drawing fluid into and forcing fluid from the fluid cavity within the barrel, the plunger further including a thumb pad comprising a flange that extends transversely from a longitudinal orientation of the plunger and includes a dimension that is greater than the proximal end opening of the barrel, the method comprising:
facilitating receipt of fluid into a fluid cavity within the barrel by moving the distal end of the plunger toward the proximal end of the barrel;
facilitating withdrawal of fluid from the fluid cavity within the barrel by moving the distal end of the plunger toward the distal end of the barrel; and
forcing the plunger with thumb pad into the barrel via the proximal end opening in the barrel such that the thumb pad flange flexes and bends and is at least partially received in the barrel so as to substantially prevent or frustrate efforts by a user to remove the plunger from the barrel.

15. The method of claim 14, wherein the syringe further includes a retractable needle assembly with a portion of the needle assembly configured to retract within the syringe after a single use, and the forcing of the plunger with thumb pad into the barrel occurs after retraction of the portion of the needle assembly into the syringe.

16. A method of utilizing a syringe of the type including a hollow barrel with an opening at a proximal end of the barrel, an opening at a distal end of the barrel and a fluid chamber disposed within the barrel, a hollow plunger extending into the barrel via the proximal end opening and axially movable within the barrel toward and away from the distal end opening, the plunger including an end wall that is releasably secured to the plunger at an opening disposed at a distal end of the plunger, and a needle assembly secured within and at a distal end of the barrel, the plunger further including a thumb pad comprising a flange that extends transversely from a longitudinal orientation of the plunger and includes a dimension that is greater than the proximal end opening of the barrel, the needle assembly including a needle holder, a resilient member biasing the needle holder toward the proximal end opening of the barrel, a retaining member releasably secured to the needle holder via a reduced material section and a needle secured to the needle holder, the method comprising:
facilitating aspiration of fluid through the needle and into a fluid cavity within the barrel by moving the distal end of the plunger toward the proximal end of the barrel;
facilitating withdrawal of the aspirated fluid from the fluid cavity within the barrel by moving the distal end of the plunger toward the distal end of the barrel;
facilitating retraction of the needle holder within a retraction cavity defined within the plunger upon complete movement of the plunger into the barrel, wherein the plunger is configured to engage the needle holder upon complete movement of the plunger into the barrel such that the end wall of the plunger is forced within the plunger and the retaining member is forced to break away and separate from the needle holder along the reduced material section to facilitate retraction into the retraction cavity of the end wall, the needle holder and the needle secured to the needle holder into the retraction cavity; and
after retraction of the needle holder within the retraction cavity, forcing the plunger with thumb pad into the barrel via the proximal end opening in the barrel such that the thumb pad flange flexes and bends and is at least partially received in the barrel so as to substantially prevent or frustrate efforts by a user to remove the plunger from the barrel.
